(19) United States
(12) Patent Application Publication
(10) Pub. No.: US 2017/0196868 A1
(43) Pub. Date: Jul. 13, 2017

(54) METHODS AND COMPOSITIONS FOR TREATMENT OF HER-POSITIVE CANCERS

(71) Applicant: UNIVERSITY OF SOUTH CAROLINA, Columbia, SC (US)

(72) Inventors: Martina MCDERMOTT, Columbia, SC (US); Igor B RONINSON, Lexington, SC (US); Eugenia BROUDE, Lexington, SC (US)

(73) Assignee: UNIVERSITY OF SOUTH CAROLINA, Columbia, SC (US)

(21) Appl. No.: 15/316,635

(22) PCT Filed: Dec. 10, 2015
(86) PCT No.: PCT/US2015/035037

§ 371 Date: Dec. 6, 2016

Related U.S. Application Data
(60) Provisional application No. 62/010,074, filed on Jun. 10, 2014.

(51) Int. Cl. A61K 31/517; A61K 39/395; A61K 31/5377

(52) U.S. Cl. CPC A61K 31/517; A61K 31/5377; A61K 39/39558; A61K 39/3955; A61K 2039/505

(57) ABSTRACT

Cancers that overexpress tyrosine kinase receptors of HER family are treated with drugs acting on these receptors. Although HER-targeting drugs have revolutionized the treatment of HER-positive cancers, high rates of primary and treatment-emergent resistance limit their clinical utility. The present inventors have now discovered that combining HER-targeting drugs with a selective inhibitor of CDK8/19 greatly improves the efficacy of such drugs, offering an improved approach to the treatment of HER-positive cancers.

### BACKGROUND OF THE INVENTION

Field of the Invention

The invention relates to the treatment of HER-positive cancers, in particular breast and colon cancers.

Summary of the Related Art

The American Cancer Society estimated that about 232,340 new cases of invasive breast cancer and about 64,640 new cases of carcinoma in situ would be diagnosed in women in 2013 in the US, and about 39,620 women would die from breast cancer. Approximately 25%-30% of breast tumors overexpress human epidermal growth factor receptor 2 (HER2/Neu, a.k.a. ERBB2, a transmembrane tyrosine kinase receptor of the HER family). HER2 positivity before the advent of HER2-directed therapy predicted a poor clinical outcome, but the availability of HER2-targeting agents has significantly improved the survival of HER2+ patients. The approved anti-HER2 agents include trastuzumab (Herceptin®), lapatinib, pertuzumab, and ado-trastuzumab emtansine. Although trastuzumab has revolutionized the treatment of HER2-positive breast cancer, high rates of primary and treatment-emergent resistance are among the barriers to improving long-term outcomes. To overcome these issues, many research efforts are devoted to developing novel anti-HER2 combination regimens, which have the potential to reduce resistance as well as the need for chemotherapy, improving the clinical outcome for patients with HER2-positive disease (Gradishar, 2013, and references therein).

Other HER2-related cancer drug targets of the HER family include EGFR (HER1, erbB1), HER3 (erbB3), and HER4 (erbB4). In particular, the epithelial growth factor receptor (EGFR) has been targeted by many approved and experimental drugs, such as erlotinib, gefitinib, afatinib and brigatinib used for lung cancer, and cetuximab used for colon cancer, as well as others (reviewed in Cheng et al., 2014).

CDK8 (ubiquitously expressed), along with its closely related isoform CDK19 (which is expressed in only a subset of tissues), is an oncogenic transcription-regulating kinase (Xu and Ji, 2011; Galbraith et al., 2010; Firestein and Hahn, 2009). In contrast to better-known members of the CDK family (such as CDK1, CDK2, and CDK4/6), CDK8 plays no role in cell cycle progression. A key function of CDK8 is phosphorylation of the C-terminal domain (CTD) of RNA polymerase II, allowing for the elongation of transcription. CTD phosphorylation by CDK8, however, is needed only for signal-induced newly initiated transcription, whereas other kinases (CDK7 and CDK9) phosphorylate CTD at the majority of genes that are already active. Hence, CDK8 inhibition affects primarily changes in the transcriptional program with little impact on ongoing transcription (Galbraith et al., 2010, 2013). CDK8 knockout in embryonic stem cells prevents embryonic development (Westerling et al., 2007) due to its essential role in the pluripotent stem cell phenotype (Adler et al., 2012), but CDK8 depletion does not inhibit the growth of normal cells (Westerling et al., 2007; Firestein et al., 2008). Furthermore, CDK8 inhibitors are neither cytotoxic nor cytostatic to normal cells or to most of the tested tumor cell types (Porter et al., 2012), which distinguishes them from almost all of the approved and experimental cancer agents. Instead, the role of CDK8 in cancer is due to its unique function as a regulator of several transcriptional programs involved in carcinogenesis (Xu and Ji, 2011) and chemotherapeutic drug response (Porter et al., 2012). CDK8 has been identified as an oncogene in melanoma (Kapoor et al., 2010) and colon cancer (Firestein et al., 2008), the CDK8 gene being amplified in ˜50% of colon cancers.

At least 75% of breast cancers express estrogen receptor (ER), a steroid hormone receptor that regulates transcription and promotes proliferation of ER-positive (ER+) breast cancers. PCT/US14/18678 teaches that CDK8/19 inhibition inhibits the transcriptional activity and mitogenic effect of ER. When tested in ER+HER2+ breast cancer cells, CDK8/19 inhibitors showed a synergistic interaction with HER2 inhibitors, lapatinib and a biosimilar of trastuzumab. This synergistic interaction was interpreted as being driven by the effect of CDK8/19 inhibitors on ER, suggesting utility for combining CDK8/19 and HER2-targeting drugs in ER+HER2+ breast cancers but not in ER-negative (ER−) HER2+ breast cancers.

### BRIEF SUMMARY OF THE INVENTION

The present inventors have tested the effect of CDK8/19 inhibitors in ER-HER2+ breast cancer cells and found, surprisingly, that such inhibitors have a synergistic effect with HER2-targeting drugs in these cells, including those that are resistant to HER2-targeting drugs. The same synergistic effect was also observed in HER2+ colon cancers. Combining CDK8/19 inhibitors with drugs targeting HER2 or EGFR also prevented the development of resistance to the latter drugs. Hence, combining drugs targeting tyrosine kinase receptors of the HER family with CDK8/19 inhibitors should be beneficial for the treatment of different HER-positive cancers. Other HER2-related cancer drug targets of the HER family include HER3 (erbB3), and HER4 (erbB4).

In a first aspect, the invention provides a method for treating a subject having a cancer that is positive for a tyrosine kinase receptor of HER family. The method comprises administering to the subject an effective amount of a selective inhibitor of CDK8/19 in combination with a drug targeting a tyrosine kinase receptor of HER family.

In some embodiments, the cancer that is positive for a tyrosine kinase receptor of HER family is an estrogen receptor negative (ER−) HER2/Neu-positive (HER2+) cancer. In these embodiments, the method comprises administering to the subject an effective amount of a selective inhibitor of CDK8/19 in combination with a HER2-targeting drug.

In some embodiments, the cancer that is positive for a tyrosine kinase receptor of HER family is an EGFR positive (EGFR+) cancer. In these embodiments, the method comprises administering to the subject an effective amount of a selective inhibitor of CDK8/19 in combination with an EGFR-targeting drug.

In some embodiments, the selective inhibitor of CDK8/19 has the structural formula I or II:

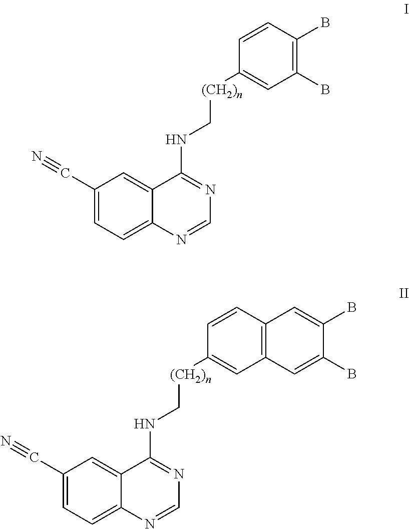

wherein each B is independently hydrogen or

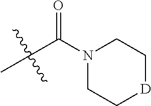

provided that at least one B is hydrogen and not more than one B is hydrogen;
D is selected from —NH, —N-lower alkyl, or O; and n is 0-2.

In a second aspect, the invention provides a pharmaceutical composition comprising a selective inhibitor of CDK8/19, a drug targeting a tyrosine kinase receptor of HER family, and a pharmaceutically acceptable carrier.

In some embodiments, the drug targeting a tyrosine kinase receptor of HER family is a HER2-targeting drug. In some embodiments, the drug targeting a tyrosine kinase receptor of HER family is an EGFR-targeting drug.

In some embodiments, the selective inhibitor of CDK8/19 has the structural formula I or II:

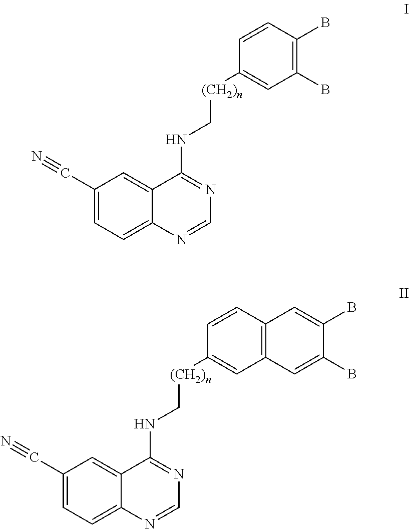

wherein each B is independently hydrogen or

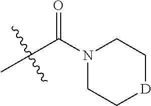

provided that at least one B is hydrogen and not more than one B is hydrogen;
D is selected from —NH, —N-lower alkyl, or O; and n is 0-2.

### BRIEF DESCRIPTION OF THE DRAWINGS

FIG. 1 shows an analysis of the interaction between CDK8/19 inhibitor Senexin B and a HER2-specific monoclonal antibody (HER, biosimilar to trastuzumab) in BT474 cells, an ER+HER2+ breast cancer cell line.

FIG. 2 shows an analysis of the interaction between CDK8/19 inhibitor Senexin B and lapatinib (LAP), a small-molecule inhibitor of both HER2 and EGFR in the ER+HER2+BT474 breast cancer cell line.

FIG. 3 shows an analysis of the interaction between CDK8/19 inhibitor Senexin B and the HER2-specific monoclonal antibody in the ER-HER2+ breast cancer cell line SKBR3.

FIG. 4 shows an analysis of the interaction between CDK8/19 inhibitor Senexin B and lapatinib in the ER-HER2+ breast cancer cell line SKBR3.

FIG. 5 shows an analysis of the interactions between CDK8/19 inhibitor Senexin B and the same HER2-specific monoclonal antibody (here designated TRAST) and lapatinib in ER-HER2+ breast cancer HCC-1419 cells, which are intrinsically trastuzumab resistant.

FIG. 6 shows an analysis of the interactions between CDK8/19 inhibitor Senexin B and the HER2-specific monoclonal antibody and lapatinib in ER-HER2+ breast cancer JIMT-1 cells, which are intrinsically trastuzumab and lapatinib resistant.

FIG. 7 shows an analysis of the interactions between CDK8/19 inhibitor Senexin B and the HER2-specific monoclonal antibody and lapatinib in ER-HER+ breast cancer SKBR3 cells and their derivative SKBR3-LT selected by 4-month culture in the presence of 250 nM lapatinib.

FIG. 8 shows an analysis of the interaction between CDK8/19 inhibitor Senexin B and lapatinib in the HER2+ colon cancer cell lines HCT-116 and HT-29.

FIG. 9 shows the effect of CDK8/19 inhibitor Senexin B on the development of lapatinib resistance in the ER+HER2+BT474 breast cancer cell line (macroscopic staining).

FIG. 10 shows the effect of CDK8/19 inhibitor Senexin B on the development of lapatinib resistance in the ER-HER2+ breast cancer cell line SKBR3 (macroscopic staining).

FIG. 11 shows the effect of CDK8/19 inhibitor Senexin B on the development of resistance to EGFR inhibitor erlotinib in the ER+HER2+BT474 breast cancer cell line (macroscopic staining).

FIG. 12 shows the effect of CDK8/19 inhibitor Senexin B on the development of resistance to EGFR inhibitor erlotinib in the ER+HER2+BT474 breast cancer cell line (microscopic imaging, three random fields per condition).

FIG. 13 shows certain selective inhibitors of CDK8/19 that are useful in the method and composition according to the invention.

### DETAILED DESCRIPTION OF THE PREFERRED EMBODIMENTS

The invention relates to the treatment of cancers positive for a tyrosine kinase receptor of HER family. The invention provides methods and formulations for combining a specific inhibitor of CDK8/19 and a drug targeting a tyrosine kinase receptor of HER family. In some embodiments the drug targeting the tyrosine kinase receptor of HER family is a HER2- or EGFR-targeting drug to treat HER2+ or EGFR+ cancers. Other HER2-related cancer drug targets of the HER family include HER3 (erbB3), and HER4 (erbB4).

In a first aspect, the invention provides a method for treating a subject having a cancer that is positive for a tyrosine kinase receptor of HER family. The method comprises administering to the subject an effective amount of a selective inhibitor of CDK8/19 in combination with a drug targeting a tyrosine kinase receptor of HER family.

In some embodiments, the cancer that is positive for a tyrosine kinase receptor of HER family is an estrogen receptor negative (ER−) HER2/Neu-positive (HER2+) cancer. In these embodiments, the method comprises administering to the subject an effective amount of a selective inhibitor of CDK8/19 in combination with a HER2-targeting drug. In some embodiments, the HER2-targeting drug is selected from the group consisting of trastuzumab (Herceptin®), lapatinib, pertuzumab, and ado-trastuzumab emtansine. These drugs include biosimilars or generics thereof.

In some embodiments, the cancer that is positive for a tyrosine kinase receptor of HER family is an EGFR positive (EGFR+) cancer. In these embodiments, the method comprises administering to the subject an effective amount of a selective inhibitor of CDK8/19 in combination with an EGFR-targeting drug. In some embodiments, the EGFR targeting drug is selected from the group consisting of erlotinib, gefitinib, afatinib, brigatinib, and cetuximab. These drugs include biosimilars or generics thereof.

For purposes of the invention a selective inhibitor of CDK8/19 is a small molecule compound that inhibits one or more of CDK8 and CDK19 to a greater extent than it inhibits certain other CDKs. In some embodiments, such compounds further inhibit CDK8/19 to a greater extent than CDK9. In preferred embodiments, such greater extent is at least 2-fold more than CDK9. A “small molecule compound” is a molecule having a formula weight of about 800 Daltons or less. The term “in combination with” means that two different agents may be administered in any order, including simultaneous administration, as well as temporally spaced order from a few seconds up to several days apart.

Some selective inhibitors of CDK8/19 useful in the methods according to the invention have been described in US Patent Publication number 20140038958. In some embodiments, the selective inhibitor of CDK8/19 has the structural formula I or II:

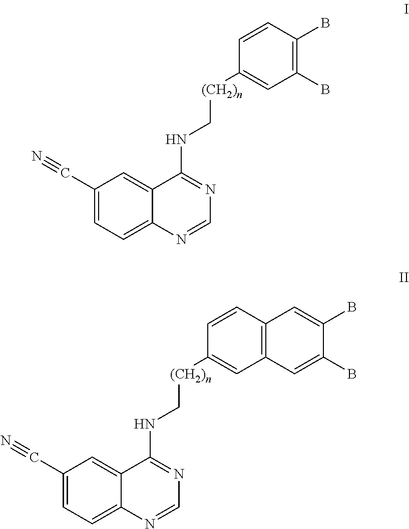

wherein each B is independently hydrogen or

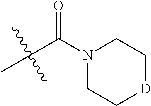

provided that at least one B is hydrogen and not more than one B is hydrogen;
D is selected from —NH, —N-lower alkyl, or O; and n is 0-2. In some embodiments lower alkyl is methyl. In some embodiments n is 0 or 1.

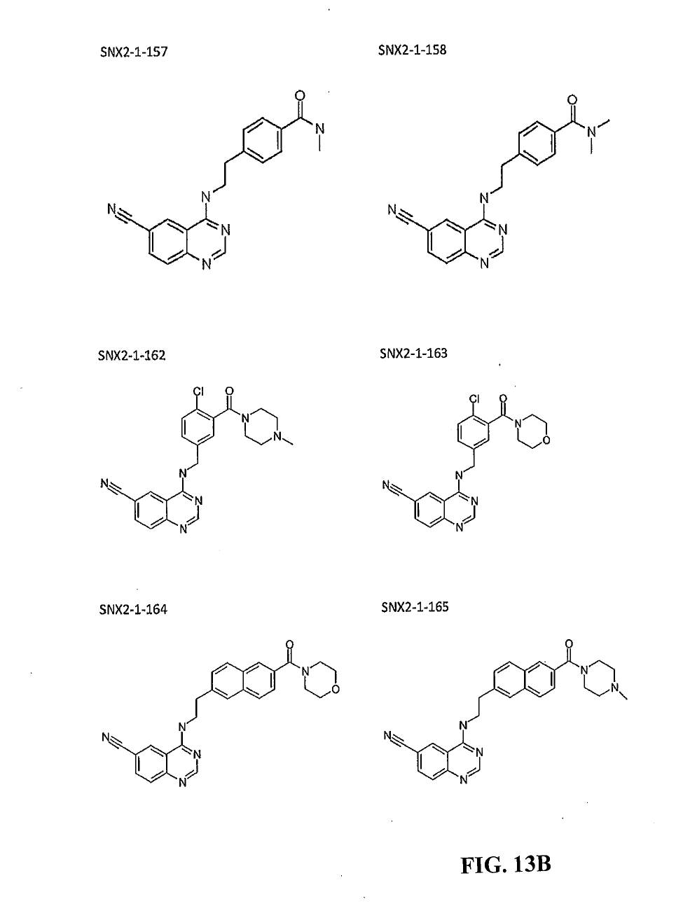

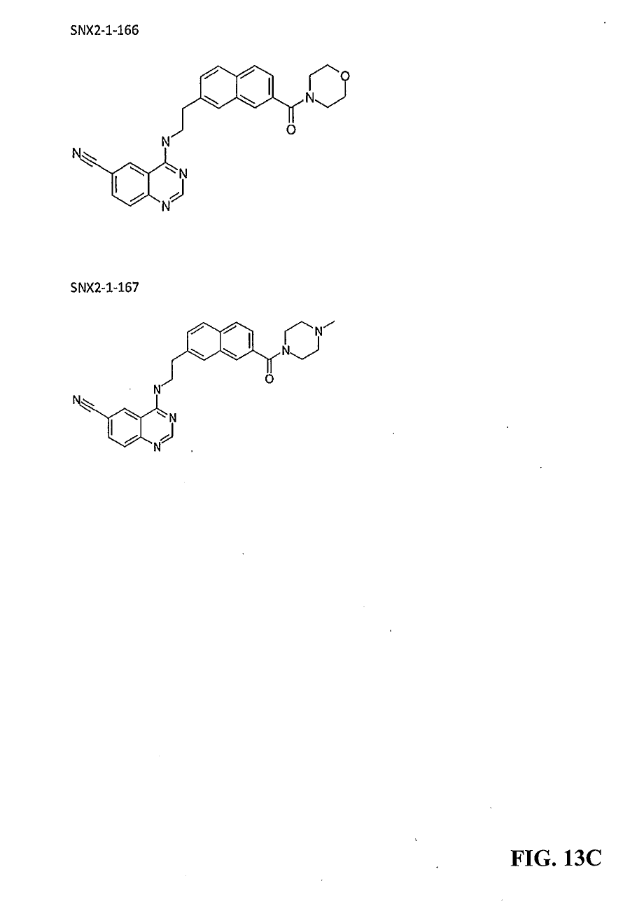

In some embodiments the selective inhibitor of CDK8/19 is selected from the group consisting of SNX2-1-162, SNX2-1-163, SNX2-1-164, SNX2-1-165, SNX2-1-166 and SNX2-1-167. In some embodiments, the selective inhibitor of CDK8/19 is SNX2-1-165. In some embodiments, the selective inhibitor of CDK8/19 is selected from the compounds shown in FIG. 13.

The active compounds are included separately or together in a pharmaceutically acceptable carrier or diluent in an amount sufficient to deliver to a patient a therapeutically effective amount without causing serious toxic effects in the patient treated. A “therapeutically effective amount” is an amount sufficient to alleviate or eliminate signs or symptoms of the disease. The effective dosage range of the pharmaceutically acceptable derivatives can be calculated based on the weight of the parent compound to be delivered. If the derivative exhibits activity in itself, the effective dosage can be estimated as above using the weight of the derivative, or by other means known to those skilled in the art. In certain applications, an effective dose range for a 70 kg patient is from about 50 mg per patient per day up to about 10 grams per patient per day, or the maximum tolerated dose. In certain preferred embodiments the dose range is from about 200 mg per patient per day to about 10 g per patient per day. In certain preferred embodiments the dose range is from about 200 mg per patient per day to about 5 g per patient per day. The dose in each patient may be adjusted depending on the clinical response to the administration of a particular drug. Administration of the pharmaceutical formulations in the methods according to the invention may be by any medically accepted route, including, without limitation, parenteral, oral, sublingual, transdermal, topical, intranasal, intratracheal, or intrarectal. In certain preferred embodiments, compositions of the invention are administered parenterally, e.g., intravenously in a hospital setting. In certain other preferred embodiments, administration may preferably be by the oral route.

In a second aspect, the invention provides a pharmaceutical composition comprising a selective inhibitor of CDK8/19, a drug targeting a tyrosine kinase receptor of HER family, and a pharmaceutically acceptable carrier.

In some embodiments, the drug targeting a tyrosine kinase receptor of HER family is a HER2-targeting drug. In some embodiments, the HER2-targeting drug is selected from the group consisting of trastuzumab (Herceptin®), lapatinib, pertuzumab, and ado-trastuzumab emtansine. These drugs include biosimilars or generics thereof.

In some embodiments, the drug targeting a tyrosine kinase receptor of HER family is an EGFR-targeting drug. In some embodiments, the EGFR targeting drug is selected from the group consisting of erlotinib, gefitinib, afatinib, brigatinib, and cetuximab. These drugs include biosimilars or generics thereof.

In some embodiments, the selective inhibitor of CDK8/19 has the structural formula I or II:

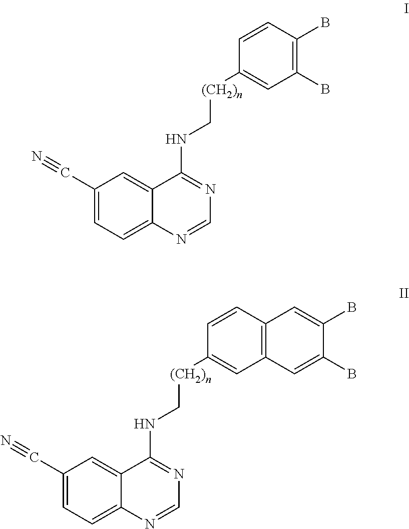

wherein each B is independently hydrogen or

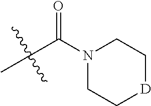

provided that at least one B is hydrogen and not more than one B is hydrogen;
D is selected from —NH, —N-lower alkyl, or O; and n is 0-2. In some embodiments lower alkyl is methyl. In some embodiments n is 0 or 1.

In some embodiments the selective inhibitor of CDK8/19 is selected from the group consisting of SNX2-1-162, SNX2-1-163, SNX2-1-164, SNX2-1-165, SNX2-1-166 and SNX2-1-167. In some embodiments, the selective inhibitor of CDK8/19 is SNX2-1-165. In some embodiments, the selective inhibitor of CDK8/19 is selected from the compounds shown in FIG. 13.

Such compositions comprise the compounds, which may be in the form of a free acid, salt or prodrug, in a pharmaceutically acceptable diluent (including, without limitation, water), carrier, or excipient. Such compositions are well known in the art and are described, e.g., in Remington's Pharmaceutical Sciences, 18th Edition, ed. A. Gennaro, Mack Publishing Co., Easton, Pa., 1990. The characteristics of the carrier will depend on the route of administration. As used herein, the term “pharmaceutically acceptable” means a non-toxic material that is compatible with a biological system such as a cell, cell culture, tissue, or organism, and that does not interfere with the effectiveness of the biological activity of the active ingredient(s). Thus, compositions according to the invention may contain, in addition to the inhibitor, diluents, fillers, salts, buffers, stabilizers, solubilizers, and other materials well known in the art. As used herein, the term “pharmaceutically acceptable salts” refers to salts that retain the desired biological activity of the above-identified compounds and exhibit minimal or no undesired toxicological effects. Examples of such salts include, but are not limited to, salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, palmoic acid, alginic acid, polyglutamic acid, naphthalenesulfonic acid, naphthalenedisulfonic acid, methanesulfonic acid, p-toluenesulfonic acid and polygalacturonic acid. The compounds can also be administered as pharmaceutically acceptable quaternary salts known by those skilled in the art, which specifically include the quaternary ammonium salt of the formula —NR+Z—, wherein R is hydrogen, alkyl, or benzyl, and Z is a counterion, including chloride, bromide, iodide, —O-alkyl, toluenesulfonate, methylsulfonate, sulfonate, phosphate, or carboxylate (such as benzoate, succinate, acetate, glycolate, maleate, malate, citrate, tartrate, ascorbate, benzoate, cinnamoate, mandeloate, benzyloate, and diphenylacetate).

The following examples are intended to further illustrate certain preferred embodiments of the invention as are not intended to limit the scope of the invention.

### Example 1

### CDK8/19 Inhibition has a Synergistic Effect with HER2/Neu Inhibition in ER+HER2+ and ER-HER2+ Breast Cancers

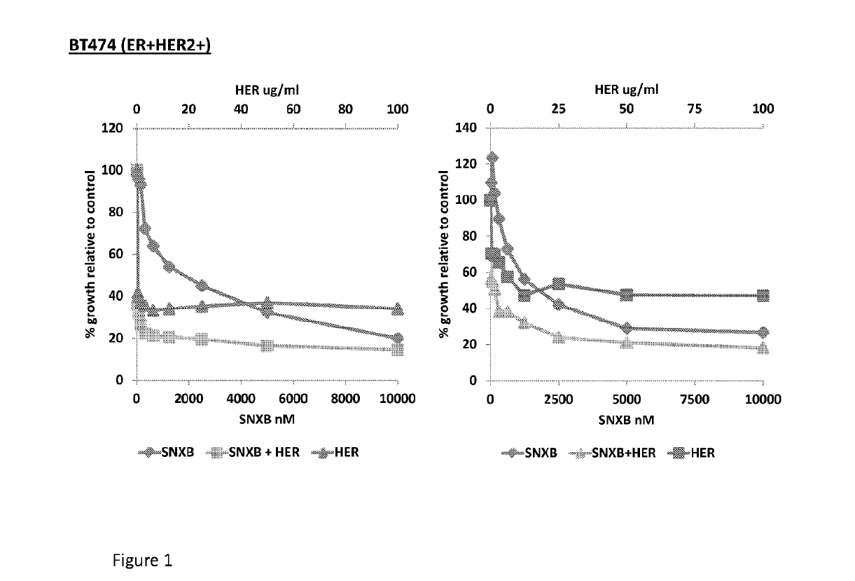

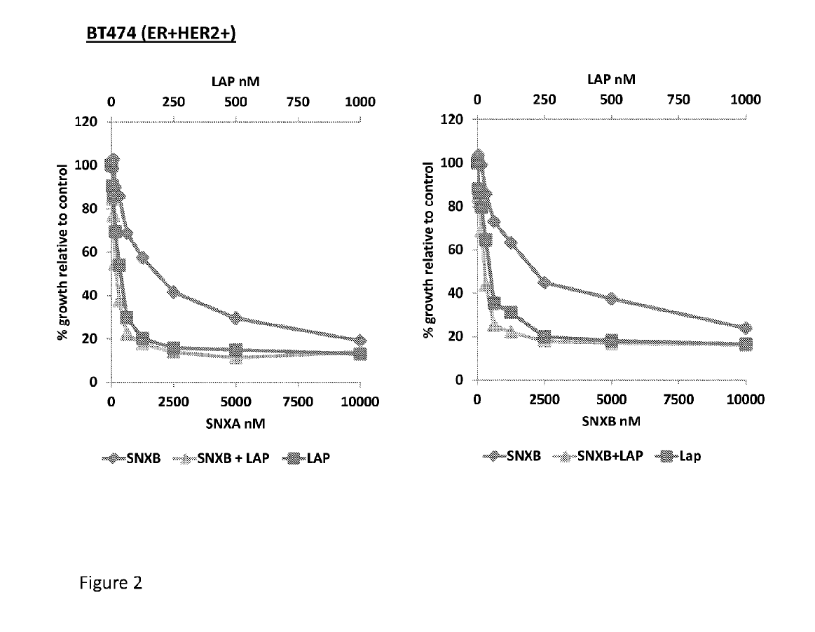

FIG. 1 presents an analysis of the nature of the interaction between CDK8/19 inhibitor Senexin B (a.k.a. SNX2-1-165) developed and owned by Senex Biotechnology, Inc.) and a HER2-specific monoclonal antibody (a biosimilar of trastuzumab from Biocad, Strelna, Russia) in BT474, a ER+HER2+ breast cancer cell line. In these experiments, 2000 cells/well were plated in each well of 96 well plates. After 24 hours, cells were treated with the indicated concentrations of Senexin B (SNXB) or a trastuzumab biosimilar (HER), alone or at a constant fixed ratio of 100 nM SNXB:1 μg/ml HER. Treatment was repeated after 3 days, and the MTT assay for relative cell number was performed after the total of 7 days of treatment. The Combination Index (CI) values were calculated using CompuSyn® (www.combosyn.com), a web tool based on the principles described in Chou (2006). This analysis showed very strong synergy between SNXB and HER in these cells. In the first experiment, the IC50 CI was infinitely low and the IC75 CI was 0.019, and in the second experiment the IC50 CI was 0.089 (FIG. 1). Similar analysis was conducted for the interaction between Senexin B and lapatinib (LAP), a small-molecule inhibitor of both HER2 and EGFR, in the same cells; using a SNXB:LAP molar ratio of 10:1. In this case, the CI values indicated strong synergy between the two compounds in the first experiment (IC50 CI=0.512) and moderate synergy in the second experiment (IC50 CI=0.728) (FIG. 2).

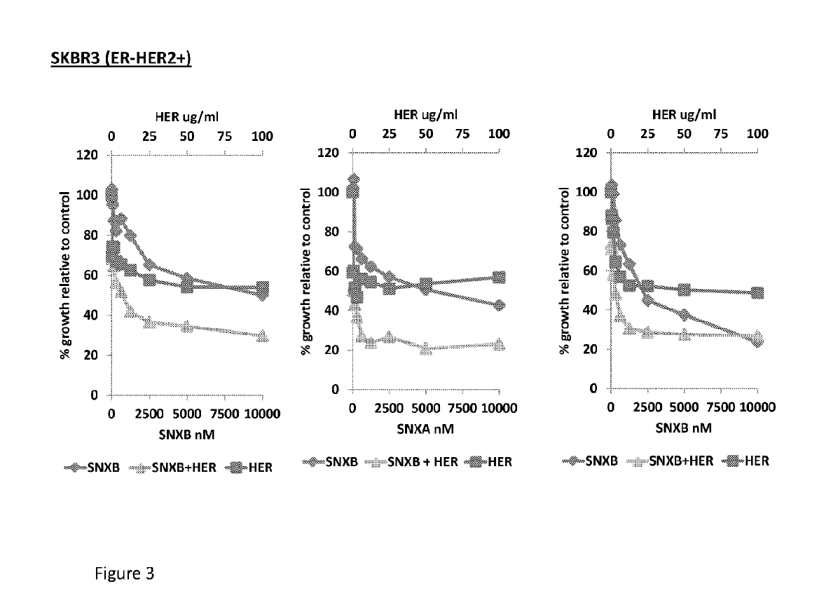

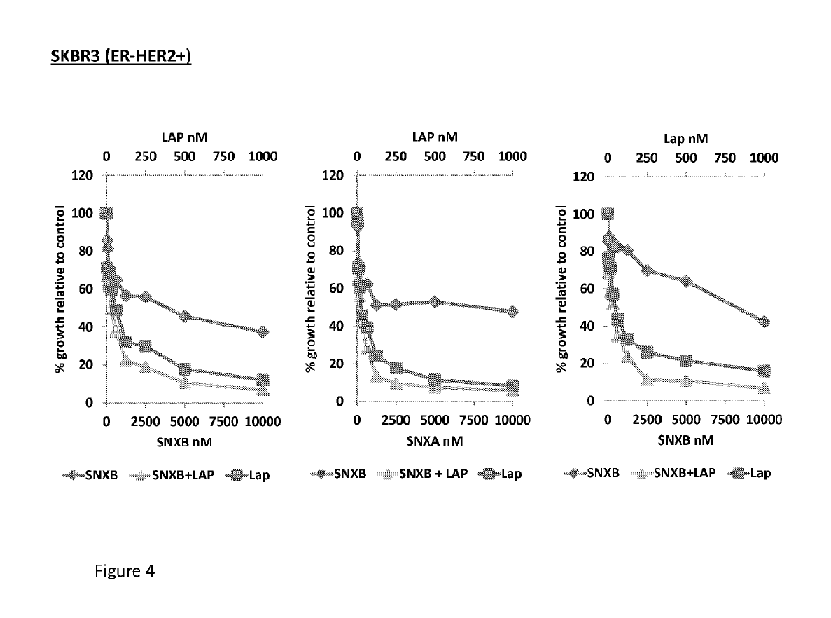

The same analysis was then conducted using an ER-HER2+ cell line SKBR3 (cells were plated at 1,500 cells per well). Surprisingly, despite the lack of ER in this cell line, Senexin B showed strong to very strong synergy with HER in three experiments (IC50 CI values were 0.107, 0.013 and 0.246) (FIG. 3). Senexin B also showed a synergy with lapatinib in SKBR3 cells (IC50 CI values were 0.611, 0.674 and 0.470 in three experiments) (FIG. 4).

These results suggest that combining a CDK8/19 inhibitor with HER2-targeting drugs is beneficial for the treatment of both ER+HER2+ and ER-HER2+ breast cancers.

### Example 2

### CDK8/19 Inhibition has a Synergistic Effect with HER2/Neu Inhibition in HER2+ Breast Cancers that are Resistant to HER2-Targeting Drugs

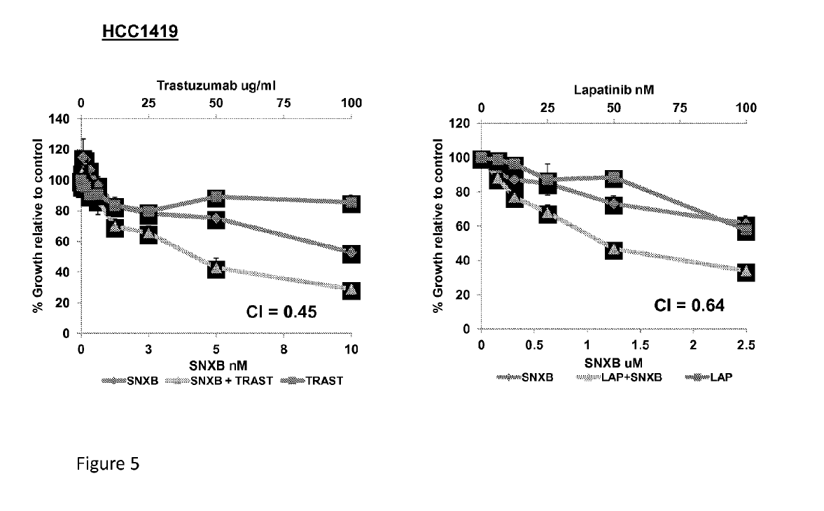

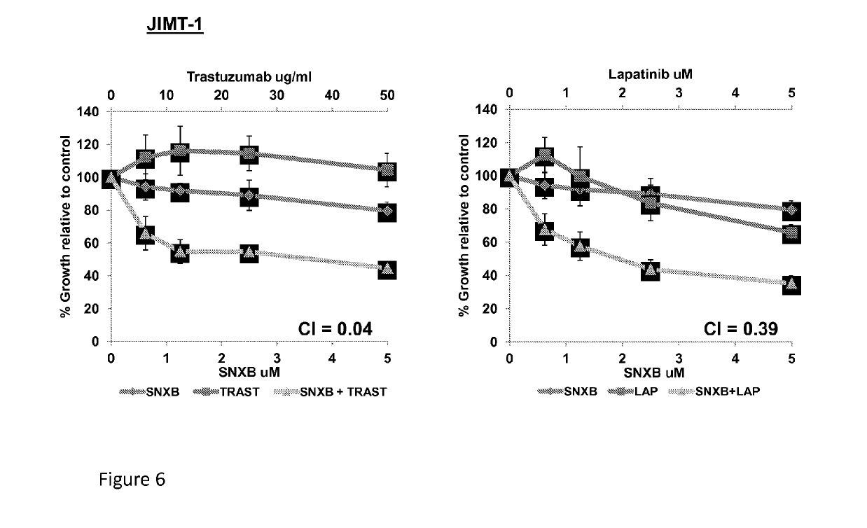

The same analysis as above was conducted in ER-HER2+ breast cancer cell line HCC-1419 which, despite high levels of HER2, is intrinsically resistant to trastuzumab but sensitive to lapatinib; this pattern of resistance has been associated with activated phosphoinositide 3-kinase/AKT signaling (O'Brien et al., 2010). We also analyzed ER-HER2+ breast cancer cell line JIMT-1, which is intrinsically resistant to both trastuzumab and lapatinib. As shown in FIG. 5 (for HCC-1419, plated at 3000 cells per well) and FIG. 6 (for JIMT-1, plated at 1500 cells per well), these cells are indeed resistant to the trastuzumab biosimilar (TRAST) and in the case of JIMT-1 cells resistant to lapatinib (LAP), but the addition of Senexin B to trastuzumab or lapatinib synergistically inhibits their growth. For HCC-1419, the CI values were 0.45 for trastuzumab and 0.64 for lapatinib (FIG. 5). For JIMT-1, IC50 CI values were 0.04 for trastuzumab and 0.39 for lapatinib (FIG. 6). These results demonstrate that CDK8/19 inhibition overcomes innate resistance to HER2-targeted drugs.

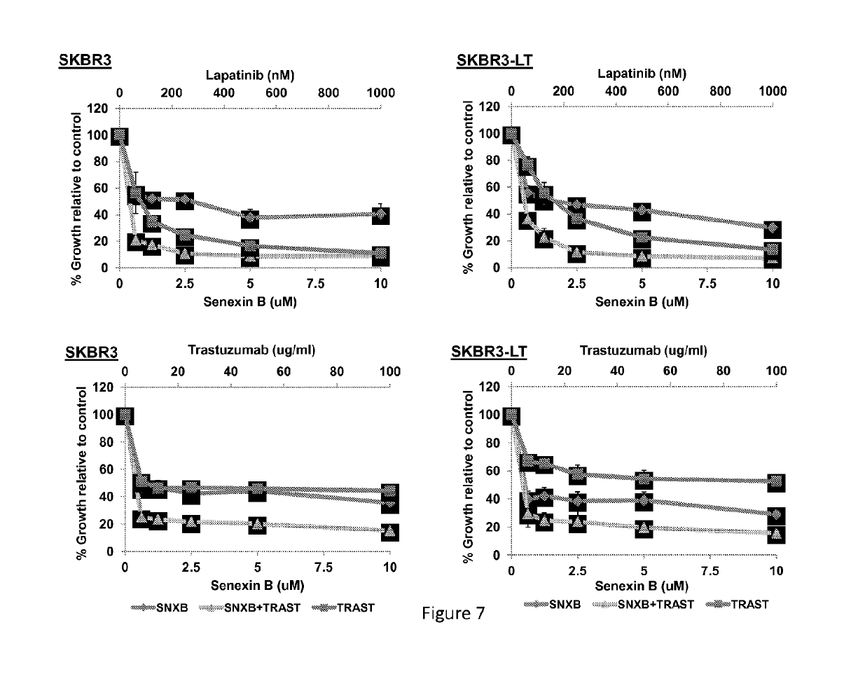

To determine if CDK8/19 inhibition has an effect on acquired resistance to HER2-targeted drugs, we selected SKBR3 ER-HER2+ breast cancer cells for lapatinib resistance, through 4-month continuous exposure to 250 nM lapatinib. The parental SKBR3 cells and the lapatinib-selected SKBR3-LT cells were then tested for resistance to HER2-targeted drugs, Senexin B, and their combinations, by plating at 1,500 cells per well in 96-well plates and then treating with Senexin B (0-10 μM), lapatinib (0-1000 nM), trastuzumab biosimilar (0-100 μg/ml) or combinations of Senexin B and lapatinib or Senexin B and trastuzumab biosimilar at a fixed ratio for 7 days. As shown in FIG. 7, SKBR3-LT cells were less sensitive to both lapatinib and trastuzumab relative to SKBR3, but their sensitivity to Senexin B alone or to Senexin B combinations with lapatinib or trastuzumab was essentially unchanged, indicating that CDK8/19 inhibition overcomes the acquired resistance to HER2-targeted drugs.

The above results demonstrate that combining CDK8/19 inhibitors with HER2-targeted drugs is beneficial for those patients whose HER2+ tumors are resistant to HER2-targeted drugs.

### Example 3

### CDK8/19 Inhibition has a Synergistic Effect with HER2/Neu Inhibition in HER2+ Colon Cancers

The importance of HER2 as a drug target is not limited to breast cancer, as HER2 gene amplification is also observed in 6-10% of colon cancers at diagnosis (Lee et al., 2014; Seo et al., 2014). A much greater number of colon cancers show immunohistochemistry-based HER2 overexpression. In contrast to breast cancers, HER2 in CRC is usually cytoplasmic, and only ˜5% of colon cancers overexpress HER2 on the cell surface, making HER2 in such cells accessible to cell-permeable small-molecule HER2 inhibitors (such as lapatinib) but not to anti-HER2 antibodies, such as trastuzumab (Blok et al., 2013).

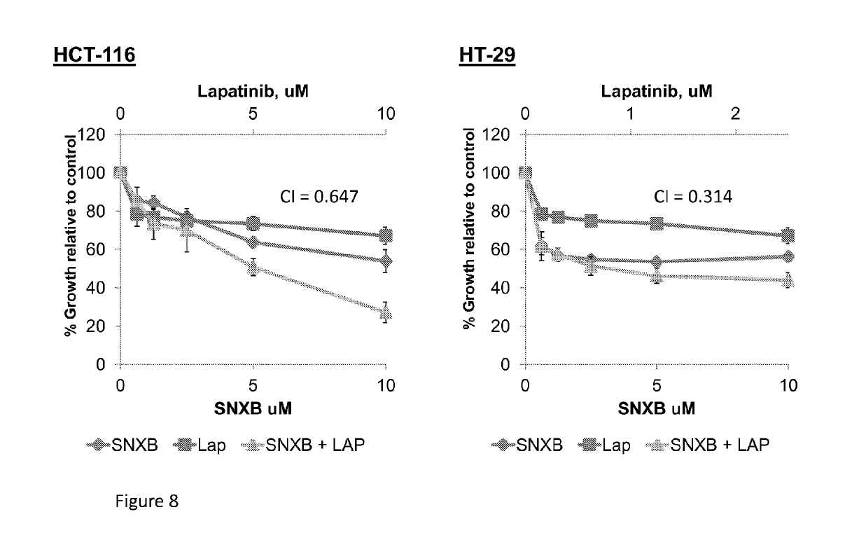

To determine if CDK8/19 inhibition affects the response of HER2+ colon cancer cells to lapatinib, we conducted the same type of analysis as above on two HER2+ colon cancer cell lines, HCT-116 and HT-29 (both plated at 1500 cells per well). As shown in FIG. 8, both of these cell lines showed only a weak response to Senexin B or lapatinib alone but a significant response to the combination of Senexin B and lapatinib (CI=0.647 for HCT-116 and 0.314 for HT-29). Hence, the combination of CDK8/19- and HER2-targeting drugs is beneficial not only to breast cancers but also to other HER2+ cancers, such as colon cancer.

### Example 4

### CDK8/19 Inhibition Prevents the Emergence of Resistance to HER2- and EGFR-Targeting Drugs

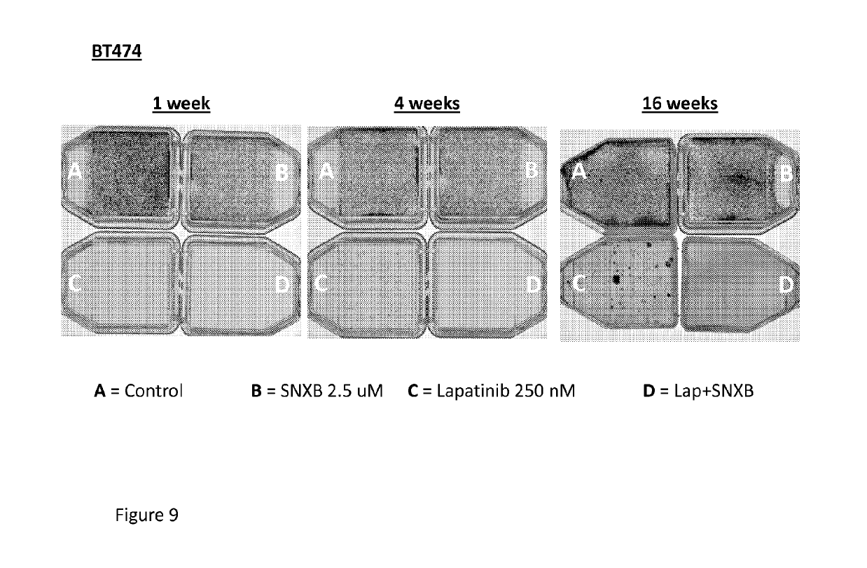

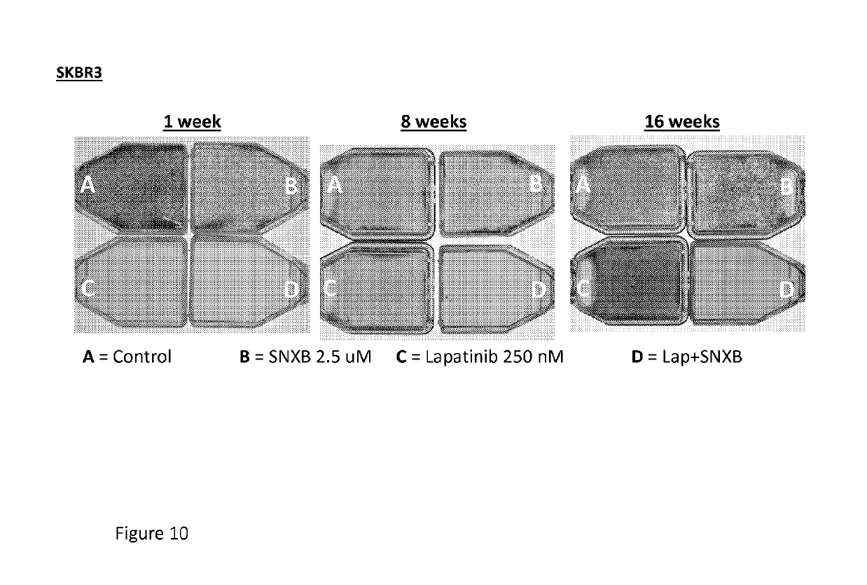

A major problem for chemotherapy in general and for molecularly targeted drugs in particular is the development of drug resistance. The ability to prevent the development of drug resistance could allow sustained response to targeted drugs and transform the outcome of cancer therapy. Since CDK8 is a key mediator of transcriptional reprogramming (Galbraith et al., 2010, 2013), we hypothesized that CDK8 inhibition may prevent the induction of transcription that may be associated with epigenetic acquisition of drug resistance. To test this hypothesis, we plated BT474 (ER+HER2+) and SKBR3 (ER-HER2+) breast cancer cell lines at 250,000 cells per T25 flask, and the cells were continuously exposed to 2.5 μM Senexin B, 250 nM lapatinib, alone and in combination, for a period of 16 weeks, changing drug-containing media twice a week. At selected time points, cells in flasks were fixed with methanol:acetic acid and stained with crystal violet. The results for BT474 are shown in FIG. 9 and for SKBR3 in FIG. 10. After 1 week of treatment, lapatinib was extremely effective as a single agent at inhibiting the growth of both cell lines, whereas Senexin B as a single agent had a limited effect. However, by week 4 (in the case of BT474) or week 8 (in the case of SKBR3), the outgrowth of cells treated with lapatinib alone became noticeable compared to cells treated with lapatinib+Senexin B combination. After 16 weeks, both cell lines treated continuously with lapatinib were actively proliferating despite the continued presence of lapatinib, however cells cultured in a combination of lapatinib and Senexin B did not grow (FIGS. 9, 10). Hence, the addition of Senexin B prevented the emergence of lapatinib resistance in both ER+ and ER−HER2-positive cell lines.

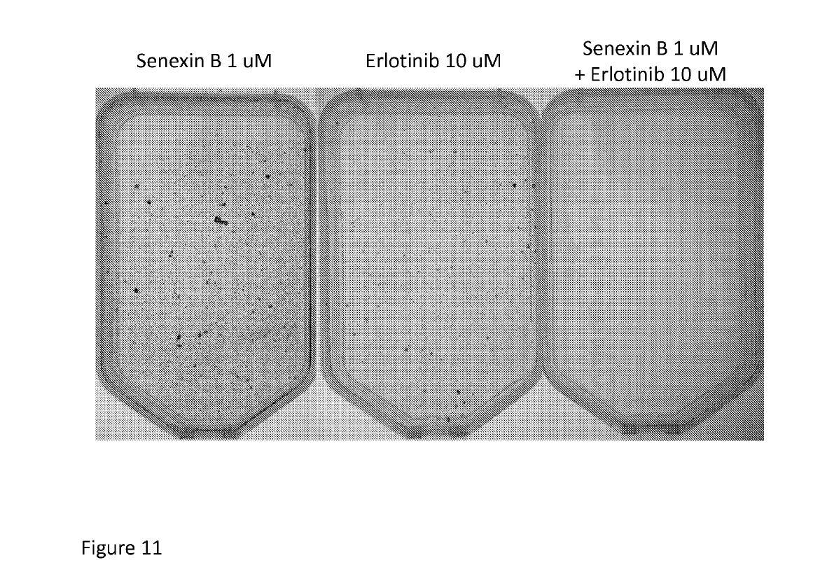

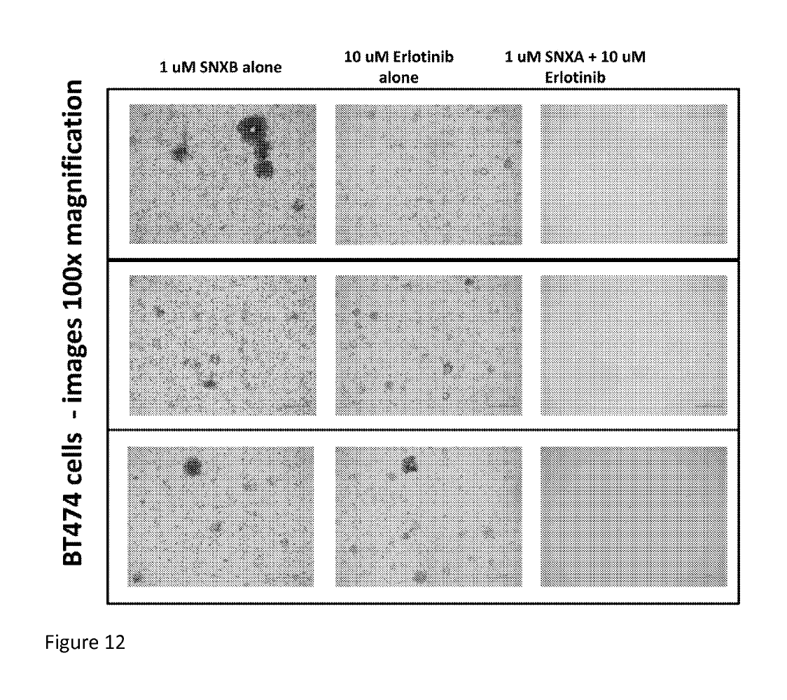

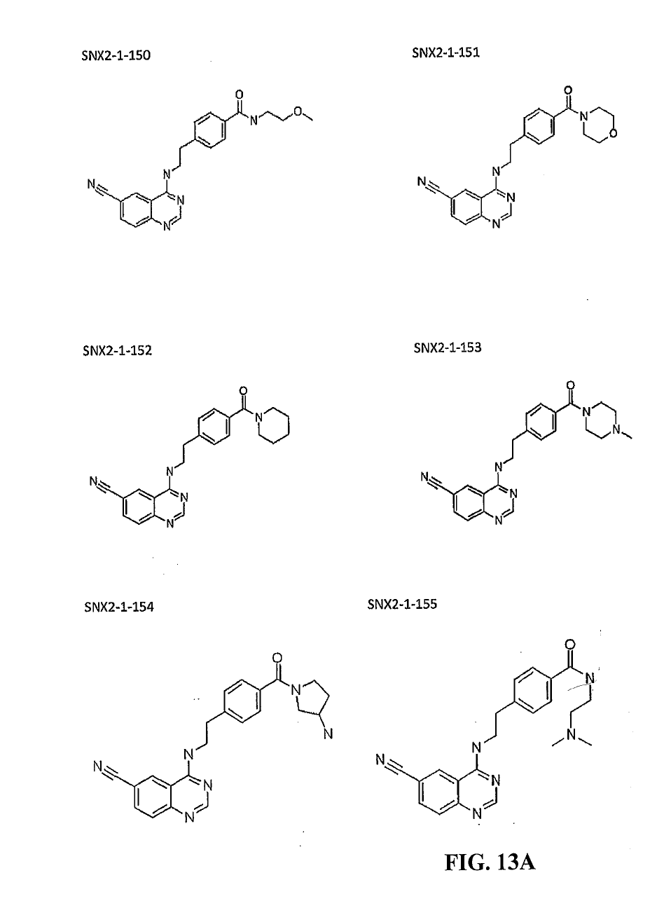

We conducted a similar experiment with BT474 breast cancer cells continuously treated with erlotinib, an inhibitor of EGFR, another member of the same HER family as HER2/Neu. 600,000 cells per T75 flask were plated in the continuous presence of 10 mM erlotinib, 1 mM Senexin B, or a combination of both (changing drug-containing media twice a week). While erlotinib alone initially inhibited cell growth, erlotinib-resistant colonies emerged after 35 days of treatment with erlotinib alone but not with an erlotinib-Senexin B combination. The macroscopic and microscopic views of the corresponding samples after 35 days of treatment are shown in FIG. 11 and FIG. 12, respectively. Hence, the addition of Senexin B prevented the emergence of resistance to EGFR inhibitor erlotinib.

The above results demonstrate that CDK8/19 inhibition prevents the development of resistance to drugs targeting different members of the HER tyrosine kinase receptor family, HER2/Neu and EGFR. This effect indicates the advisability of combining drugs targeting receptors of this family with CDK8/19 inhibitors.

### REFERENCES

Blok, E. J., Kuppen, P. J., van Leeuwen, J. E., & Sier, C. F. (2013) Cytoplasmic Overexpression of HER2: a Key Factor in Colorectal Cancer. Clin. Med. Insights. Oncol. 7, 41-51.
Cheng, W., Hu, Y., Sheng, R. (2014). Development of EGFR family small molecule inhibitors for anticancer intervention: an overview of approved drugs and clinical candidates. Curr Med Chem. 21, 4374-4404.
Chou, T. C. (2006). Theoretical basis, experimental design, and computerized simulation of synergism and antagonism in drug combination studies. Pharmacol Rev 58, 621-681.
Firestein, R., Bass, A. J., Kim, S. Y., Dunn, I. F., Silver, S. J., Guney, I., Freed, E., Ligon, A. H., Vena, N., Ogino, S., Chheda, M. G., Tamayo, P., Finn, S., Shrestha, Y., Boehm, J. S., Jain, S., Bojarski, E., Mermel, C., Barretina, J., Chan, J. A., Baselga, J., Tabernero, J., Root, D. E., Fuchs, C. S., Loda, M., Shivdasani, R. A., Meyerson, M., and Hahn, W. C. (2008). CDK8 is a colorectal cancer oncogene that regulates beta-catenin activity. Nature 455, 547-551.
Firestein, R. and Hahn, W. C. (2009). Revving the Throttle on an oncogene: CDK8 takes the driver seat. Cancer Res 69, 7899-7901.
Galbraith, M. D., Donner, A. J., and Espinosa, J. M. (2010). CDK8: a positive regulator of transcription. Transcription. 1, 4-12.
Galbraith, M. D., Allen, M. A., Bensard, C. L., Wang, X., Schwinn, M. K., Qin, B., Long, H. W., Daniels, D. L., Hahn, W. C., Dowell, R. D. et al. (2013) HIF1A employs CDK8-mediator to stimulate RNAPII elongation in response to hypoxia. Cell 153, 1327-1339.
Gradishar, W. J. (2013). Emerging approaches for treating HER2-positive metastatic breast cancer beyond trastuzumab. Annals of Oncology 24: 2492-2500.
Kapoor, A., Goldberg, M. S., Cumberland, L. K., Ratnakumar, K., Segura, M. F., Emanuel, P. O., Menendez, S., Vardabasso, C., Leroy, G., Vidal, C. I., Polsky, D., Osman, I., Garcia, B. A., Hernando, E., and Bernstein, E. (2010). The histone variant macroH2A suppresses melanoma progression through regulation of CDK8. Nature 468, 1105-1109.
Lee, W. S., Park, Y. H., Lee, J. N., Baek, J. H., Lee, T. H., & Ha, S. Y. (2014) Comparison of HER2 expression between primary colorectal cancer and their corresponding metastases. Cancer Med. 3, 674-680.
O'Brien, N. A. Browne, B. C., Chow, L., Wang, Y., Ginther, C., Arboleda, J., Duffy, M. J., Crown, J., O'Donovan, N., and Slamon, D. J. (2010). Activated Phosphoinositide 3-Kinase/AKT Signaling Confers Resistance to Trastuzumab but not Lapatinib. Mol Cancer Ther. 9, 1489-1502.
Porter, D. C., Farmaki, E., Altilia, S., Schools, G. P., West, D. K., Chen, M., Chang, B. D., Puzyrev, A. T., Lim, C. U., Rokow-Kittell, R., Friedhoff, L. T., Papavassiliou, A. G., Kalurupalle, S., Hurteau, G., Shi, J., Baran, P. S., Gyorffy, B., Wentland, M. P., Broude, E. V., Kiaris, H., and Roninson, I. B. (2012). Cyclin-dependent kinase 8 mediates chemotherapy-induced tumor-promoting paracrine activities. Proc. Natl. Acad. Sci. U.S.A. 109, 13799-13804.
Seo, A. N., Kwak, Y., Kim, D. W., Kang, S. B., Choe, G., Kim, W. H., & Lee, H. S. (2014) HER2 Status in Colorectal Cancer: Its Clinical Significance and the Relationship between HER2 Gene Amplification and Expression. PLoS. One. 9, e98528.
Xu, W. and Ji, J. Y. (2011). Dysregulation of CDK8 and Cyclin C in tumorigenesis. J. Genet. Genomics 38, 439-452.

What is claimed is:

1. A method for treating a subject having a cancer that expresses a tyrosine kinase receptor of HER family, comprising administering to the subject an effective amount of a selective inhibitor of CDK8/19 in combination with a drug targeting the tyrosine kinase receptor of HER family.

2. The method according to claim 1 wherein the subject has estrogen receptor negative (ER−) HER2/Neu-positive (HER2+) cancer.

3. The method according to claim 1 or 2, wherein the cancer is breast cancer.

4. The method according to claim 1 or 2, wherein the cancer is colon cancer.

5. The method according to claim 1, 2, 3, or 4, wherein the drug targeting the tyrosine kinase receptor of HER family is a HER-2 targeting drug.

6. The method according to claim 5, wherein the HER-2 targeting drug is selected from the group consisting of trastuzumab (Herceptin®), lapatinib, pertuzumab, and ado-trastuzumab emtansine.

7. The method according to claim 1, 3, or 4, wherein the subject has epithelial growth factor receptor (EGFR)-positive cancer.

8. The method according to claim 7, wherein the drug targeting the tyrosine kinase receptor of HER family is an EGFR-targeting drug.

9. The method according to claim 8, wherein the EGFR-targeting drug is selected from the group consisting of erlotinib, gefitinib, afatinib, brigatinib, and cetuximab.

10. The method according to claim 1, 2, 3, 4, 5, 6, 7, 8, or 9, wherein the selective inhibitor of CDK8/19 has the structural formula I or II: 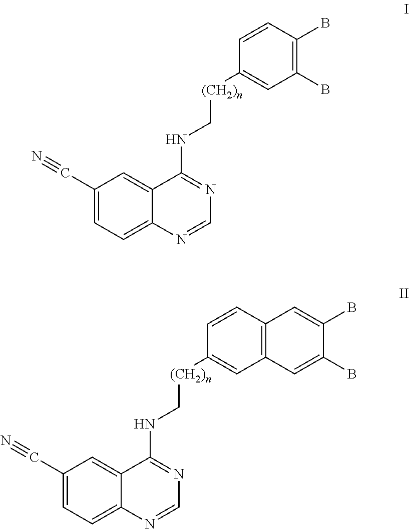
wherein each B is independently hydrogen or 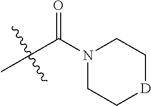
provided that at least one B is hydrogen and not more than one B is hydrogen;
D is selected from —NH, —N-lower alkyl, or O;
and n is 0-2.

11. The method according to claim 10, wherein the lower alkyl is methyl.

12. The method according to claim 10 or 11, wherein n is 0 or 1.

13. The method according to claim 10, wherein the selective inhibitor of CDK8/19 is selected from the group consisting of SNX2-1-162, SNX2-1-163, SNX2-1-164, SNX2-1-165, SNX2-1-166 and SNX2-1-167.

14. The method according to claim 13, wherein the selective inhibitor of CDK8/19 is SNX2-1-165.

15. The method according to claim 1, 2, 3, 4, 5, 6, 7, 8 or 9, wherein the selective inhibitor of CDK8/19 is selected from the compounds shown in FIG. 13.

16. A pharmaceutical composition comprising a selective inhibitor of CDK8/19, a drug targeting the tyrosine kinase receptor of HER family, and a pharmaceutically acceptable carrier.

17. The pharmaceutical composition according to claim 16, wherein the drug targeting the tyrosine kinase receptor of HER family is a HER2-targeting drug

18. The pharmaceutical composition according to claim 17, wherein the HER2-targeting drug is selected from the group consisting of trastuzumab (Herceptin®), lapatinib, pertuzumab, and ado-trastuzumab emtansine.

19. The pharmaceutical composition according to claim 16, wherein the drug targeting the tyrosine kinase receptor of HER family is an EGFR-targeting drug.

20. The pharmaceutical composition according to claim 19, wherein the EGFR-targeting drug is selected from the group consisting of erlotinib, gefitinib, afatinib, brigatinib, and cetuximab.

21. The pharmaceutical composition according to claim 16, 17, 18, 19, or 20, wherein the selective inhibitor of CDK8/19 has the structural formula I or II: 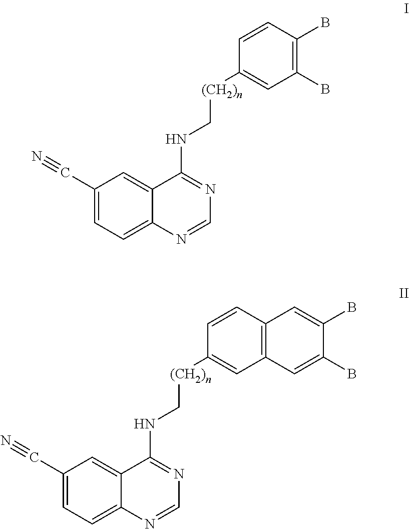
wherein each B is independently hydrogen or 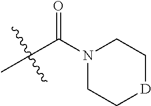
provided that at least one B is hydrogen and not more than one B is hydrogen;
D is selected from —NH, —N-lower alkyl, or O;
and n is 0-2.

22. The pharmaceutical composition according to claim 21, wherein the lower alkyl is methyl.

23. The pharmaceutical composition according to claim 21 or 22, wherein n is 0 or 1.

24. The pharmaceutical composition according to claim 21, wherein the selective inhibitor of CDK8/19 is selected from the group consisting of SNX2-1-162, SNX2-1-163, SNX2-1-164, SNX2-1-165, SNX2-1-166 and SNX2-1-167.

25. The pharmaceutical composition according to claim 24, wherein the selective inhibitor of CDK8/19 is SNX2-1-165.

26. The pharmaceutical composition according to claim 16, 17, 18, 19, or 20, wherein the selective inhibitor of CDK8/19 is selected from the compounds shown in FIG. 13.
